(19) United States
(12) Patent Application Publication
(10) Pub. No.: US 2016/0374455 A1
(43) Pub. Date: Dec. 29, 2016

(54) COSMETIC CONTAINER WITH ANTIBACTERIAL PROTECTION

(71) Applicant: GIXIA GROUP CO., Zhubei (TW)

(72) Inventors: JUNG-YA HSIEH, Hsinchu (TW); YUNG-FU LIN, Hsinchu (TW); YUAN-HSIN CHANG, Hsinchu (TW)

(21) Appl. No.: 15/165,026

(22) Filed: May 26, 2016

Related U.S. Application Data
(60) Provisional application No. 62/186,356, filed on Jun. 29, 2015.

(51) Int. Cl. A45D 40/00; A61L 2/20

(52) U.S. Cl. CPC A45D 40/0068; A61L 2/20

(57) ABSTRACT

A cosmetic container with antibacterial protection includes a bottle, a cap, a plate, a chamber, a deoxy sterilization material, and a switch mechanism. The cosmetics in the bottle can be sealed with the cap. The plate is mounted in the cap. The chamber is formed between the cap and the plate. The deoxy sterilization material is in the chamber. The switch mechanism is mounted in the cap adjacent to the plate for opening or closing the chamber, the switch mechanism opening the chamber and its contents to the contents of the cosmetic container when the cap is used to close the bottle, but closing and sealing the chamber in the cap when the cap is unscrewed so that the bacteria-killing contents are not volatilized into the air.

### CROSS REFERENCE TO RELATED APPLICATION

This application claims priority from U.S. Provisional Patent Application Ser. No. 62/186,356, filed Jun. 29, 2015.

### FIELD

The subject matter herein generally relates to safe physical storage containers, and in particular to a cosmetic container with antibacterial protection.

### BACKGROUND

There are six factors deciding the growth rate of bacteria: nutrition, minerals, oxygen, environment pH value, moisture, and temperature. Cosmetics are rich in natural oil, gelatin, nutrition components, proteins, amino acids and water, which attract bacteria from the environment and from a user's fingers. As the bacteria reproduce, they lead to deterioration in the cosmetics, such as fat rancidity, breakdown of the effective ingredients, and degeneration. The deterioration of the cosmetics may actually cause allergies to the user. In the worst situation, severe skin disease can be caused. The notorious pseudomonas aeruginosa bacteria may damage the cornea or lead to keratitis and even blindness of the user.

Antibacterial technology includes packaging materials using antibacterial agent, a carbon dioxide gas generating agent or sterilization agent, or de-oxidizing packaging. The current antibacterial technology is effective for controlling the bacterial growth in a closed environment. However, in actual use, the cosmetics are in an open environment. Contact outside air and the user's hand during use causes a rapid oxidation reaction of the deoxidizer in a cosmetic container, or causes the antibacterial agent, the carbon dioxide gas generating agent or the sterilization agent to volatilize, whereby the deoxidizer, the antibacterial agent, the carbon dioxide gas generating agent or the sterilization agent gradually lose effectiveness.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a side view of a cosmetic container of a first embodiment of the present disclosure.

FIG. 2 is a cross-sectional view of part of the cosmetic container of FIG. 1 showing a cap in a closed position.

FIG. 3 is a cross-sectional view of part of the cosmetic container of FIG. 1 showing the cap in a to-be-opened position.

FIG. 4 is a view similar to FIG. 2 of a second embodiment of the present disclosure.

FIG. 5 is a view similar to FIG. 3 of the second embodiment of the present disclosure.

FIG. 6 is a schematic top view of a cosmetic container of a third embodiment of the present disclosure showing a chamber opened.

FIG. 7 is a schematic top view of the cosmetic container of FIG. 6 showing the chamber closed.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The present disclosure, including the accompanying drawings, is illustrated by way of examples and not by way of limitation. Several definitions that apply throughout this disclosure will now be presented. It should be noted that references to “an” or “one” embodiment in this disclosure are not necessarily to the same embodiment, and such references mean “at least one.”

The term “comprising” means “including, but not necessarily limited to”; it specifically indicates open-ended inclusion or membership in a so-described combination, group, series, and the like.

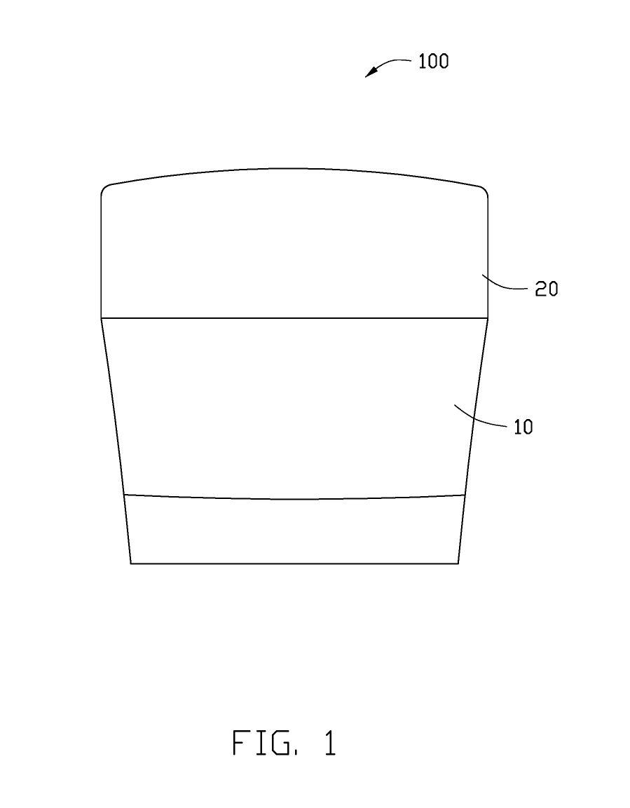

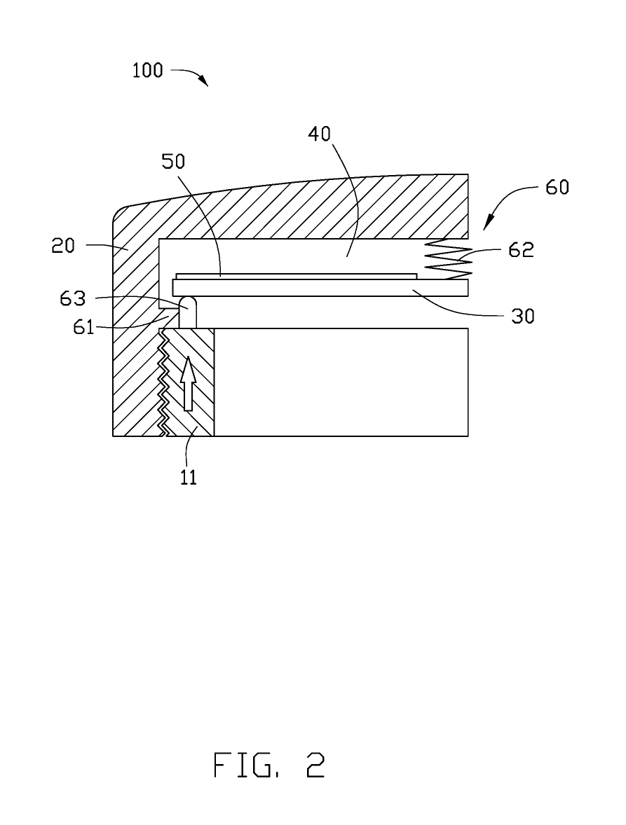

FIG. 1 shows an embodiment of a cosmetic container 100. FIG. 2 is a partial cross-sectional view of the cosmetic container 100 of FIG. 1. The cosmetic container 100 includes a bottle 10, a cap 20, a plate 30, a chamber 40, a deoxy sterilization material 50, and a switch mechanism 60. The bottle 10 is for containing cosmetics or other materials. The bottle 10 includes a cylindrical neck 11 (only a portion of which is shown in FIG. 2) having threads (not labeled) for mating with threads (not labeled) in the cap 20. The cap 20 can be screwed/unscrewed to the threaded neck 11 to close/open the bottle 10. When the bottle 10 is covered with the cap 20, the cosmetics inside the bottle 10 are sealed from the outside air. When the bottle 10 is uncovered, the cosmetics inside the bottle 10 comes into contact with bacteria in the air or on a user's hand. The plate 30 is mounted in the cap 20. The chamber 40 is formed between the cap 20 and the plate 30. The deoxy sterilization material 50 is placed in the chamber 40. The deoxy sterilization material 50 may be in the form of a coating, a block, a sheet or particles. The deoxy sterilization material 50 in the form of a coating is provided on a top surface of the plate 30 in the chamber 40. The switch mechanism 60 is mounted in the cap 20 adjacent to the plate 30 for opening or closing the chamber 40 by using 61/63 as described in more detail below.

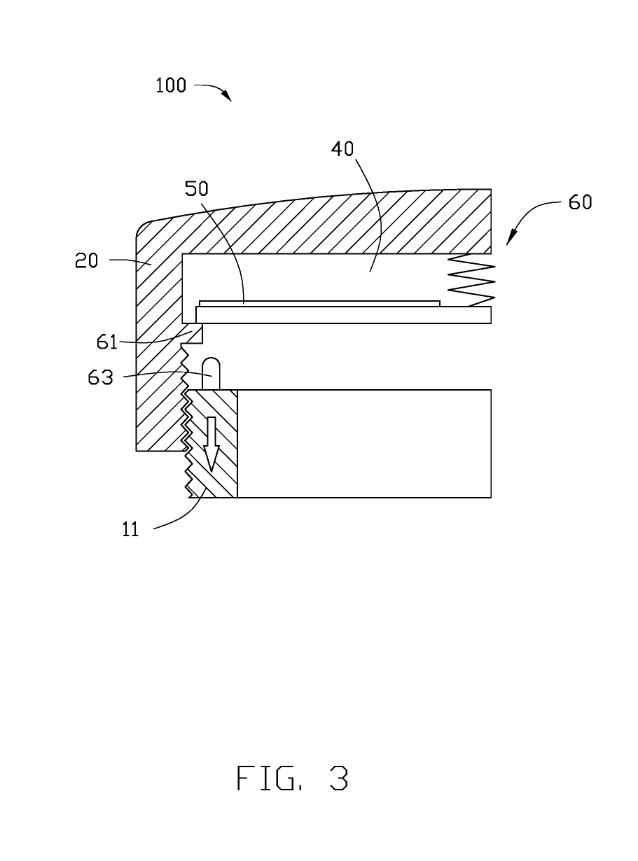

Referring to FIG. 2, when the cap 20 is screwed onto the neck 11 to close the bottle 10, the switch mechanism 60 is operated to open the chamber 40 such that vapor generated by the deoxy sterilization material 50 flows from the chamber 40 into the bottle 10, this reduces deterioration of the cosmetics by bacterial action. Referring to FIG. 3, when the cap 20 is unscrewed from the neck 11 to open the bottle 10, the switch mechanism 60 is operated to cause 61 and 63 to close, seal, and isolate the chamber 40 containing the deoxy sterilization material 50.

Referring to FIGS. 2 and 3, a first embodiment of the cosmetic container 100 shows that the switch mechanism 60 has an annular rib 61, an elastic member 62, and a protrusion 63. The annular rib 61 is formed on an inside annular surface of the cap 20. The elastic member 62, such as a spring, is mounted in the chamber 40 between the cap 20 and the plate 30. One end of the elastic member 62 is secured to an inside top surface of the cap 20 and the other end of the elastic member 62 urges against the top surface of the plate 30. The elastic member 62 is capable of exerting a pressure on the plate 30 to force it against the annular rib 61 for closing the chamber 40. The protrusion 63 is formed on a top end of the neck 11 and is capable of extending upwardly from the annular rib 61 to push the plate 30 for opening the chamber 40.

Referring to FIG. 2, when the cap 20 is screwed onto the neck 11 to close the bottle 10, the protrusion 63 extends upwardly from the annular rib 61 to push the plate 30 to open the chamber 40. Referring to FIG. 3, when the cap 20 is unscrewed from the neck 11 to open the bottle 10, the protrusion 63 effectively ceases extending upwardly from the annular rib 61. This allows the plate 30 under spring pressure from the elastic member 62 to return to exert pressure against the annular rib 61 and seal it, and the chamber 40 is automatically closed.

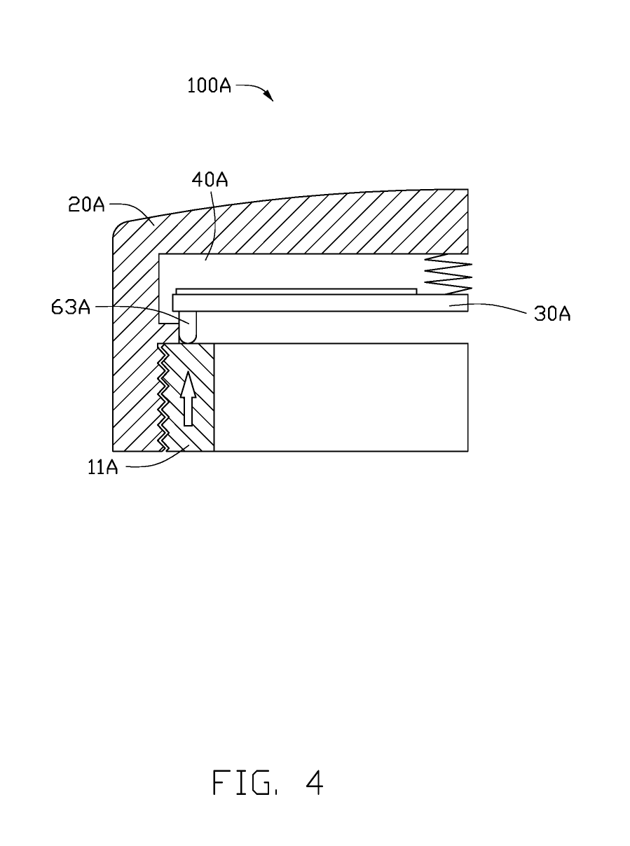

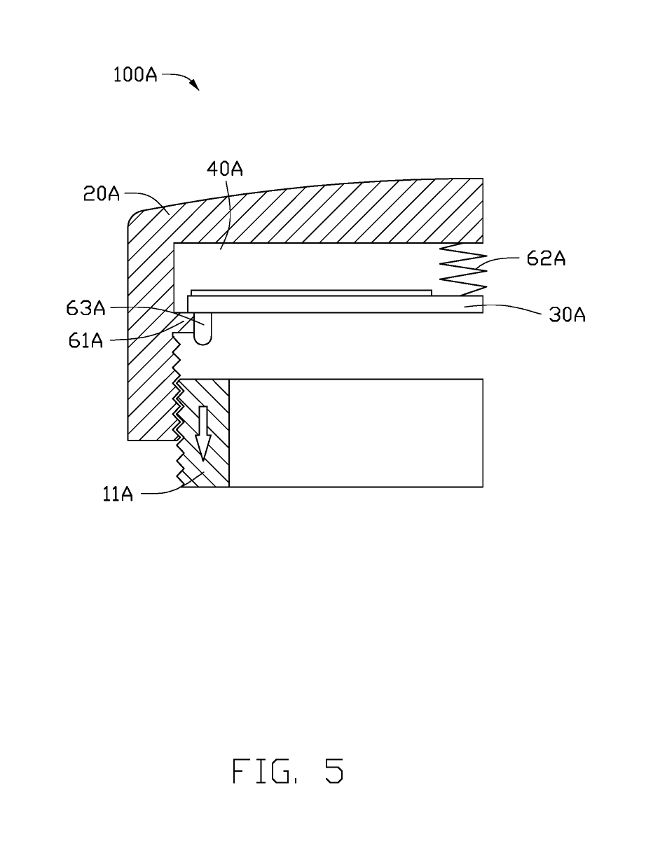

Referring to FIGS. 4 and 5, there is shown a second embodiment of the cosmetic container 100A. The second embodiment is the same as the first embodiment, except that the protrusion 63A is formed on a bottom surface of the plate 30A. The protrusion 63A is capable of moving upwardly by abutting against the top end of the neck 11A for opening the chamber 40A. Referring to FIG. 4, when the cap 20A is screwed onto the neck 11A, the protrusion 63A moves upwardly by abutting against the top end of the neck 11A such that the plate 30A also moves upwardly to open the chamber 40. Referring to FIG. 5, when the cap 20A is unscrewed from the neck 11A, the protrusion 63A is effectively withdrawn, such that the plate 30A returns to abut against the annular rib 61A by the pressure exerted by the elastic member 62A, and the chamber 40A is automatically closed.

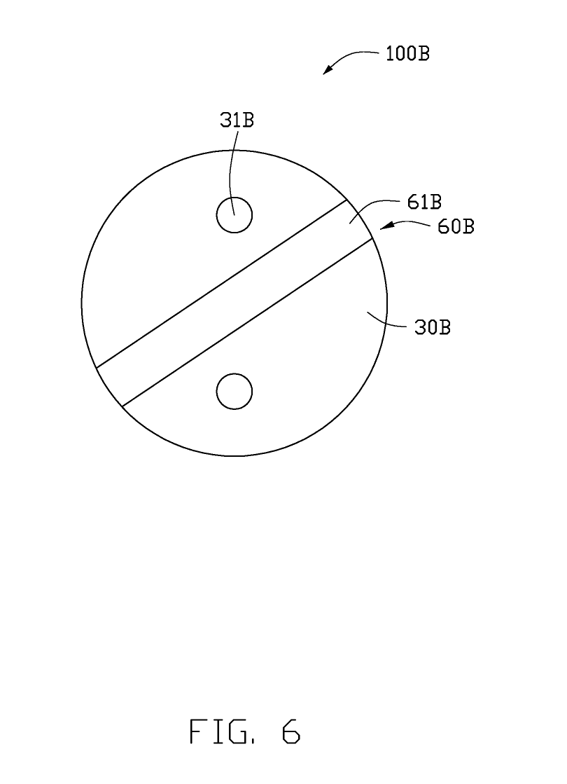

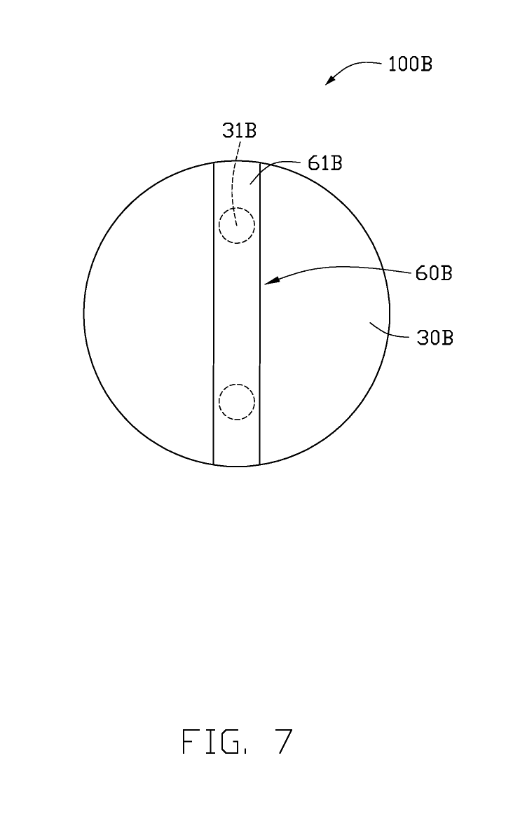

Referring to FIGS. 6 and 7, there is shown a third embodiment of the cosmetic container 100B. The plate 30B has at least one through hole 31B communicating with the chamber (not labeled). Preferably, the plate 30B has two diametrically opposite through holes 31B. The switch mechanism 60B has an elongated block 61B rotatably mounted on the plate 30B. The elongated block 61B is capable of being rotated to cover or uncover the through holes 31B of the plate 30B for closing or opening the chamber.

The embodiments shown and described above are only examples. Many details are often found in the art such as the other features of a cosmetic container. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. A cosmetic container comprising:
a bottle;
a cap covering the bottle;
a plate mounted in the cap;
a chamber formed between the cap and the plate;
a deoxy sterilization material in the chamber; and
a switch mechanism mounted in the cap adjacent to the plate and operated to open or close the chamber.

2. The cosmetic container of the claim 1, wherein the switch mechanism has
an annular rib formed on an inside annular surface of the cap;
an elastic member mounted in the chamber between the cap and the plate and capable of exerting a pressure on the plate to force it to abut against the annular rib for closing the chamber; and
a protrusion capable of opening the chamber.

3. The cosmetic container of the claim 2, wherein the protrusion is formed on a top end of the bottle and capable of extending from the annular rib to push the plate for opening the chamber.

4. The cosmetic container of the claim 3, wherein the deoxy sterilization material is in the form of a coating and is provided on a top surface of the plate.

5. The cosmetic container of the claim 4, wherein one end of the elastic member is secured to an inside top surface of the cap and the other end of the elastic member is secured to the top surface of the plate.

6. The cosmetic container of the claim 4, wherein the bottle includes a neck having threads for mating with threads in the cap.

7. The cosmetic container of the claim 5, wherein the bottle includes a neck having threads for mating with threads in the cap.

8. The cosmetic container of the claim 6, wherein the protrusion is formed on a top end of the neck of the bottle.

9. The cosmetic container of the claim 7, wherein the protrusion is formed on a top end of the neck of the bottle.

10. The cosmetic container of the claim 2, wherein the protrusion is formed on a bottom surface of the plate and capable of moving by abutting against a top end of the bottle for opening the chamber.

11. The cosmetic container of the claim 10, wherein the deoxy sterilization material is in the form of a coating and is provided on a top surface of the plate.

12. The cosmetic container of the claim 11, wherein one end of the elastic member is secured to an inside top surface of the cap and the other end of the elastic member is secured to the top surface of the plate.

13. The cosmetic container of the claim 11, wherein the bottle includes a neck having threads for mating with threads in the cap.

14. The cosmetic container of the claim 12, wherein the bottle includes a neck having threads for mating with threads in the cap.

15. The cosmetic container of the claim 13, wherein the protrusion is capable of moving by abutting against a top end of the neck of the bottle.

16. The cosmetic container of the claim 14, wherein the protrusion is capable of moving by abutting against a top end of the neck of the bottle.

17. The cosmetic container of the claim 1, wherein the plate has at least one through hole communicating with the chamber and the switch mechanism has an elongated block rotatably mounted on the plate and capable of covering or uncovering the through holes of the plate for closing or opening the chamber.

18. The cosmetic container of the claim 17, wherein the plate has two diametrically opposite through holes.

19. The cosmetic container of the claim 18, wherein the deoxy sterilization material is in the form of a coating and is provided on a top surface of the plate.

20. The cosmetic container of the claim 19, wherein the bottle includes a neck having threads for mating with threads in the cap.
